(19) United States
(12) Patent Application Publication
(10) Pub. No.: US 2023/0034006 A1
(43) Pub. Date: Feb. 2, 2023

(54) DENTAL TOOL HAVING A THERMOELECTRIC DEVICE

(71) Applicant: PDS Inc., North Vancouver (CA)

(72) Inventors: Mahmoud Torabinejad, Laguna Niguel, CA (US); Mason A. Bahador, Baltimore, MD (US); Mehdi Talwerdi, North Vancouver, BC (CA); Winfield S. Fisher, Maple Ridge, BC (CA); Vahid Pishvaei, Surrey, BC (CA); Bahador Sohrabi, Vancouver, BC (CA)

(73) Assignee: PDS Inc., North Vancouver, BC (CA)

(21) Appl. No.: 17/782,962

(22) PCT Filed: Dec. 4, 2020
(86) PCT No.: PCT/CA2020/051676

§ 371 Date: Jun. 6, 2022

Related U.S. Application Data
(60) Provisional application No. 62/944,666, filed on Dec. 6, 2019.

(51) Int. Cl. A61B 5/00

(52) U.S. Cl. CPC A61B 5/4827; A61B 5/7475; A61B 5/4547

(57) ABSTRACT

A dental tool electrically powered by a power source includes: a handpiece having at least one switch; and a probe attachable to the handpiece so as to define a probe tip, the dental tool being operable to maintain the probe at its probe tip at a specifiable temperature. At least one of the handpiece and the probe includes a thermoelectric device, the dental tool being operable to maintain the probe at said probe tip at a specifiable temperature in response to user actuation of the at least one switch. Optionally, a second handpiece having an electrically conductive enclosure houses a second switch held by the patient. A method of conducting a dental test on a tooth of the patient includes: generating a sensory stimulus at the probe tip when the probe tip is in contact with the tooth; and receiving an actuation by the patient of the second switch.

### BACKGROUND OF THE INVENTION

### 1. Field of Invention

This invention relates to tools for dentistry and to a dental tool having a thermoelectric device.

### 2. Description of Related Art

Dental pulp sensitivity testing involves applying a sensory stimulus to a patient's tooth and then monitoring the patient's reaction to determine the health status of the tooth pulp.

Applying the sensory stimulus may involve cooling a patient's tooth, such as by applying a pre-cooled dental tool to the tooth; or heating the patient's tooth, such as by applying a pre-heated dental tool to the tooth. However, by such known dental procedures the temperature of the sensory stimulus diminishes with time after pre-cooling or pre-heating, thus the temperature applied to the tooth cannot be known precisely nor precisely controlled.

The sensory stimulus may be an electrical current. By known procedures, however, applying an electrical current to the patient's tooth involves the use of separate equipment distinct from that used when the sensory stimulus is cold or heat.

A known dental transillumination test involves shining a light through the patient's tooth, which may reveal cracks in the tooth, tooth decay, or other dental defects. Light sources for use in dental transillumination testing are commercially available. However, commercially available transillumination testers are not capable of also performing dental pulp sensitivity tests.

A thermoelectric, or Peltier, device creates a heat flux between the junction of two different types of materials (e.g. semiconductor materials) when electrical power, such as a direct current, is applied to the device. The two different types of materials in a conventional thermoelectric device are associated with opposing sides of the thermoelectric device so that heat is moved from one side to the other side of the thermoelectric device. A thermoelectric device can provide cooling or heating at a given side relative to the temperature of the opposing side by reversing the polarity of the direct current (DC) electricity applied to the thermoelectric device.

For a given electric power applied to a thermoelectric device, a lower temperature at the cold side of the thermoelectric device can be achieved by cooling the hot side of the thermoelectric device.

An object of the invention is to address one or more of the above shortcomings.

### SUMMARY

The above shortcomings may be addressed by providing, in accordance with one aspect of the invention, a dental tool comprising: (a) a handpiece; and (b) a probe attachable to the handpiece so as to define a probe tip, the dental tool being operable to maintain the probe at its probe tip at a specifiable temperature.

The handpiece may be dimensioned for being held by a hand of a human user. The probe may be dimensioned for insertion into the mouth of a human dental patient. The probe tip may be dimensioned for contact with a tooth of the human dental patient.

The dental tool may be electrically powered by a power source. The power source may be operable to receive electrical power from an alternating current (AC) source. The AC source may have a voltage in the range of 110 VAC to 230 VAC. The AC source may have a voltage of less than 110 VAC. The AC source may have a voltage of greater than 230 VAC. The AC source may be a wall outlet source. The power source may be operable to receive electrical power from a direct current (DC) source. The DC source may be a battery. The battery may be a rechargeable battery. The dental tool may be operable to receive electrical power from the battery. The handpiece may be operable to receive electrical power from the battery. The handpiece may be dimensioned to receive the battery.

The dental tool may include electronic control circuitry. The electronic control circuitry may be operable to control the operation of the handpiece. The electronic control circuitry may be operable to control the operation of the probe. The dental tool may include a control wire connectable between the electronic control circuitry and the handpiece. The dental tool may include a control wire extending between the electronic control circuitry and the handpiece. The handpiece may include the electronic control circuitry.

The dental tool may include a first switch. The handpiece may include the first switch. The first switch may be user actuatable. The first switch may be a first button switch. The first button switch may be a first push-button switch. The dental tool may include a plurality of switches. The handpiece may include a plurality of switches. The handpiece may be operable to receive user input. The handpiece may be operable to receive the user input via the first switch.

The electronic control circuitry may be operable to control the operation of the handpiece in response to user actuation of the first switch. The electronic control circuitry may be operable to control the operation of the probe in response to user actuation of the first switch. The electronic control circuitry may be operable to produce an audio signal in response to user actuation of the first switch. The handpiece may include a user interface. The user interface may include an indicator. The indicator may be operable to display numeric data. The indicator may include a liquid-crystal display (LCD). The indicator may be operable to emit light. The indicator may include a light-emitting diode (LED). The LED may be a multi-color LED. The user interface may be operable to display a plurality of color-coded indications of status.

The probe may be attached to the handpiece. The probe may be integrally attached to the handpiece. The probe may be removably attachable to the handpiece. The probe may be fixed to the handpiece. The probe may be attachably fixed to the handpiece. The probe may be permanently attached to the handpiece. The handpiece may include a handpiece connector for receiving the probe. The probe may include a probe connector for engaging the handpiece connector. The probe connector may be disposed at a connector end of the probe. The connector end and the probe tip may be disposed at opposing ends of the probe. The probe may define the probe tip distal from the handpiece. The probe tip may be disposed distal from the handpiece when the probe is attached to the handpiece. The probe may project from the handpiece toward the probe tip.

At least one of the handpiece and the probe may include a thermoelectric device. The handpiece may include the thermoelectric device. The probe may include the thermoelectric device. The thermoelectric device may be disposed at the probe tip. The dental tool may be operable to maintain the probe tip at the specifiable temperature in response to the user actuation of the first switch. The dental tool may be operable to maintain the probe tip at the specifiable temperature in response to the user actuation of one of the plurality of switches. The electronic control circuitry may be operable to produce an audio signal in response to the specifiable temperature being sensed.

The probe may include a probe holder. The probe holder may be dimensioned to receive the probe. The probe holder may be operable to attach the received probe to the handpiece.

The handpiece may be operable to receive a plurality of different probes. The dental tool may include the plurality of different probes. Each of the different probes may be removably attachable to the handpiece. A kit may include the handpiece and the plurality of probes. The probe holder may be dimensioned to receive any one of the plurality of different probes. The probe may be a single-use disposable probe. Each of the plurality of different probes may be single-use. Each of the plurality of different probes may be disposable.

The dental tool may be operable to identify the probe that is attached to the handpiece. The handpiece may include a detector for detecting at least one of the type of probe and the probe. The probe may exhibit a pre-determined electrical parameter. The handpiece may be operable to sense the pre-determined electrical parameter. The pre-determined electrical parameter may be a resistance. The plurality of different probes may exhibit a plurality of different resistances. The probe may include a resistor exhibiting the pre-determined resistance. The probe may exhibit a pre-determined optical parameter. The handpiece may be operable to sense the pre-determined optical parameter. The pre-determined optical parameter may be a pattern of optical blockages and optical pass-throughs disposed on a projection of the probe. The handpiece may include an optical source and an optical receiver. The optical source and the optical receiver may be disposed on opposing sides of a slot. The slot may be dimensioned to receive the projection. The projection may be connectorized. The electronic control circuitry may be operable to control the identified probe in accordance with its identification. The electronic control circuitry may be operable to receive user input for overriding the identification of the identified probe. The electronic control circuitry may be operable to control the identified probe in accordance with the received user input. The electronic control circuitry may be operable to control the identified probe in accordance with the received user input instead of in accordance with its identification.

The dental tool may include a first thermoelectric device disposed adjacent the probe holder. The handpiece may include the first thermoelectric device. The thermoelectric device may be in thermal communication with the probe holder. The probe holder may be in thermal communication with the received probe. The probe may include a thermally conductive path from a base of the probe received by the probe holder to the probe tip. The thermoelectric device may be operable to adjust the temperature of the probe holder to a first specifiable temperature. The thermoelectric device may be operable to adjust the temperature of the probe to a second specifiable temperature. The thermoelectric device may be operable to adjust the temperature of the probe at its probe tip to a third specifiable temperature. The dental tool may include a probe sensor. The probe sensor may be operable to sense the temperature of the probe holder. The handpiece may include the probe sensor. The probe sensor may be operable to sense the temperature of the probe. The probe sensor may be operable to sense the temperature of the probe at its probe tip. The probe may include the probe sensor. The dental tool may include a heatsink in thermal communication with the thermoelectric device. The handpiece may include the heatsink. The heatsink and the probe holder may be disposed on opposing sides of the thermoelectric device. The heatsink and the probe holder may be disposed on the same side of the thermoelectric device. The probe tip may be disposed on an opposing side of the thermoelectric device from the probe holder. The dental tool may include a heatsink sensor for sensing the temperature of the heatsink. The handpiece may include the heatsink sensor. The dental tool may include a fan for generating an airflow at the heatsink. The handpiece may include the fan. The electrical control circuitry may be operable to control the operation of the fan. The electrical control circuitry may be operable to control a speed of the fan.

The dental tool may include a heat pipe. The heat pipe may be disposed in thermal communication between the thermoelectric device and the heatsink.

The dental tool may include a multiple-stage thermoelectric device. The multiple-stage thermoelectric device may include a plurality of thermoelectric-device stages in a stacked configuration thereof.

The dental tool may include a plurality of thermoelectric devices. The dental tool may include a pair of thermoelectric devices. The handpiece may include the pair of thermoelectric devices. The pair of thermoelectric devices may be disposed on opposing sides of the probe holder. The dental tool may be operable to sink heat at a first side of a first thermoelectric device, the opposing side of the first thermoelectric device being in thermal communication with the probe holder, and operable to sink heat at a second side of a second thermoelectric device, the opposing side of the second thermoelectric device being in thermal communication with the probe holder. The handpiece may include the first and second thermoelectric devices. The dental tool may include first and second heatsinks. The first and second heatsinks may be disposed in thermal communication with the first and second thermoelectric devices, respectively.

The dental tool may be operable to cool one side of the thermoelectric device by water cooling. The dental tool may include a water block for cooling the one side of the thermoelectric device. The water block may include water passages for circulating therethrough a liquid coolant. The dental tool may include a radiator for radiating heat away from the liquid coolant. The dental tool may be operable to circulate the liquid coolant between the water block and the radiator. The handpiece may include the water block. The handpiece may include the radiator.

The probe may be a cooling-type probe. The probe may be a heating-type probe. The probe may be straight. The probe may be angled between the connector end and the probe tip. The probe tip may be rounded. The probe tip may be blunt. The probe tip may be pointed. The probe tip may be thin. The probe may be dimensioned for insertion of at least the probe tip into a root canal of a tooth of the human dental patient.

The dental tool may include a second handpiece. The second handpiece may be dimensioned for being held by a hand of the human dental patient. The second handpiece may be in electrical communication with the electronic control circuitry. The second handpiece may include a second switch. The electronic control circuitry may be operable to control the operation of the handpiece in response to actuation of the second switch. The electronic control circuitry may be operable to control the operation of the probe in response to actuation of the second switch. The electronic control circuitry may be operable to produce an audio signal in response to actuation of the second switch.

The probe may be an electrical diagnostic probe. The dental tool may be operable to generate an electrical potential at the electrical diagnostic probe. The electrical diagnostic probe may define an electrical diagnostic probe tip distal from the connector end. The dental tool may be operable to generate an electrical potential at the electrical diagnostic probe tip. The second handpiece may include an electrically conductive enclosure. The second switch may be operable to interrupt an electrical current passing through the electrical diagnostic probe tip when the electrical diagnostic probe is in electrical communication with the second handpiece. The second switch may be operable to interrupt an electrical current passing through the electrical diagnostic probe tip when the electrical diagnostic probe is in electrical communication with the second handpiece via a tooth and a hand of the human dental patient.

The probe may be a visible light probe. The dental tool may be operable to generate visible light at the visible light probe.

The power source may include an AC (alternating current) to DC (direct current) converter. The power source may include a DC-to-DC converter.

In accordance with another aspect of the invention, there is provided a method of conducting a dental test on a tooth of a human patient. The method involves: (a) generating a sensory stimulus at a probe tip of a probe attached to a handpiece when the probe tip is in contact with the tooth; and (b) receiving an actuation by the patient of a switch of a second handpiece being held by the patient.

Step (a) may involve generating a thermal stimulus at a pre-determined temperature. Step (a) may involve generating the thermal stimulus at a pre-determined cold temperature. Step (a) may involve generating the thermal stimulus at a pre-determined hot temperature.

Step (a) may involve generating an electrical stimulus. Step (b) may involve receiving the actuation of the switch of the second handpiece having an electrically conductive enclosure. The method may further involve: (c) ceasing to generate the electrical stimulus in response to receiving the actuation of the switch of the second handpiece.

Step (a) may involve generating a visible light stimulus.

In accordance with another aspect of the invention, there is provided a dental tool for conducting a dental test on a tooth of a human patient, the dental tool comprising: (a) handpiece means for generating a sensory stimulus at the tooth; and (b) second handpiece means for receiving an actuation by the patient of a switch.

The foregoing summary is illustrative only and is not intended to be in any way limiting. Other aspects and features of the present invention will become apparent to those of ordinary skill in the art upon review of the following description of embodiments of the invention in conjunction with the accompanying figures and claims.

### BRIEF DESCRIPTION OF THE DRAWINGS

In drawings which illustrate by way of example only embodiments of the invention:

FIG. 1 is a schematic representation of a dental tool according to a first embodiment of the invention;

FIG. 2 is a schematic representation of the dental tool according to a second embodiment of the invention;

FIG. 3 is a schematic representation of a portion of the dental tool shown in FIG. 1, showing probes having identifiable probe types in accordance with two exemplary techniques for identifying probe type.

FIG. 4 is an image of two different probes having different shapes;

FIG. 5 is a schematic representation of two further different probes having different tip shapes;

FIG. 6 is an image of an exemplary thermoelectric device suitable for use in the dental tool shown in FIG. 1, showing ceramic plates on opposing sides of the thermoelectric device;

FIG. 7 is a schematic representation of variations of the dental tool shown in FIG. 1, showing different arrangements of thermoelectric devices in relation to the probe holder and the probe;

FIG. 8 is a set of different views of a portion of the dental tool of FIG. 1, showing a straight probe attached to a handpiece;

FIG. 9 is a set of different views of the portion shown in FIG. 8, showing an angled or bent probe;

FIG. 10 is a set of different views of the portions shown in FIGS. 8 and 9, showing a probe having a pointed tip;

FIG. 11 is a perspective view of a water block of a dental tool according to a third embodiment of the invention;

FIG. 12 is a perspective view of a power converter of the dental tool according to the third embodiment of the invention;

FIG. 13 is an image of a probe according to any embodiment of the dental tool, showing visible light emitted from the probe;

FIG. 14 is an image of a multi-stage thermoelectric device suitable for use in any embodiment of the dental tool, showing stacked stages of the multi-stage thermoelectric device;

FIG. 15 is a schematic representation of the dental tool according to a fourth embodiment, showing the multi-stage thermoelectric device of FIG. 14;

FIG. 16 is a perspective view of a dental tool according to the fourth embodiment, showing a gun-shaped housing;

FIG. 17 is a right-side view of the dental tool shown in FIG. 16;

FIG. 18 is a right-side sectional view of the dental tool shown in FIG. 16;

FIG. 19 is a left-side sectional view of the dental tool shown in FIG. 16;

FIG. 20 is top and bottom views of the dental tool shown in FIG. 16;

FIG. 21 is a perspective view of a dental tool according to a variation of the fourth-embodiment dental tool shown in FIG. 16, showing a display screen;

FIG. 22 is a schematic representation of the use of the dental tool according to any embodiment thereof, showing a second patient-held handpiece; and

FIG. 23 is a flow diagram of a method of operation of the dental tool according to any embodiment thereof, showing the step of generating a sensory stimulus.

### DETAILED DESCRIPTION

A dental tool for conducting a dental test on a tooth of a human patient includes: (a) handpiece means for generating a sensory stimulus at the tooth; and (b) second handpiece means for receiving an actuation by the patient of a switch.

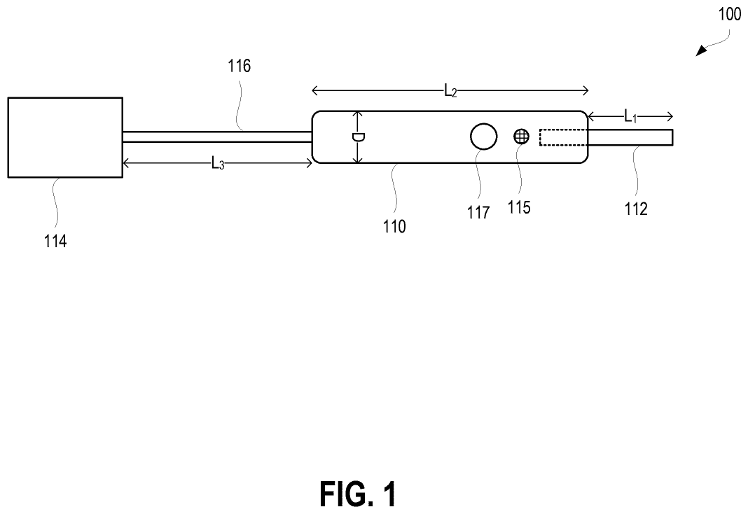

Referring to FIG. 1, the dental tool according to a first embodiment of the invention is shown generally. In some embodiments, the dental tool is a multifunction diagnostic testing tool for examining sensitivity of a tooth pulp. It covers heat, cold and electrical testing plus includes a light source to inspect and identify a cracked tooth or perform other diagnostic procedure(s).

The dental tool includes in some embodiments a handpiece, detachable probe(s), an electronic control box and a connection wire. The handpiece has opening(s) to accept different probes, a visual indicator for showing the status of operation of the dental tool, and button(s) to start and stop testing.

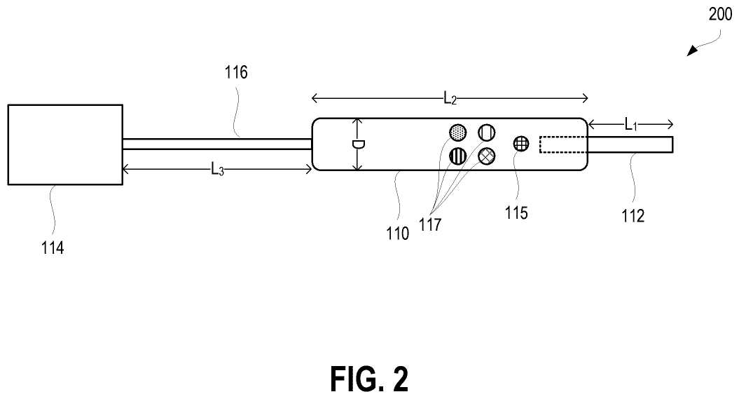

FIG. 2 shows an optional embodiment in which four separate buttons actuate and identify four different dental tool operations, which are heating, cooling, electrical testing, and transillumination light operation.

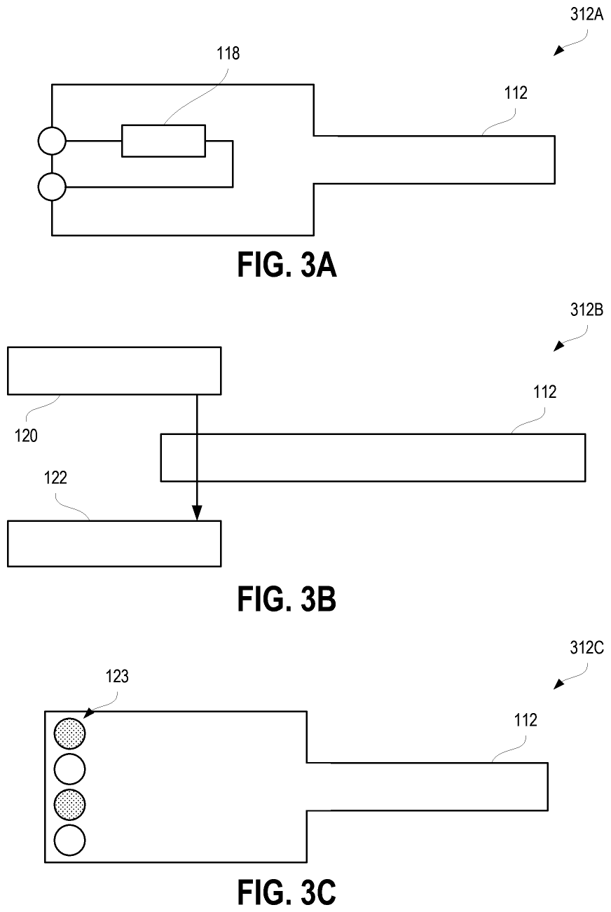

Referring back to FIG. 1 and with reference to FIG. 3, the dental tool in some embodiments automatically identifies the type of probe attached to the handpiece and its corresponding operation. In this embodiment, which may be referred to as a smart probe embodiment, each probe is coded differently according to its type. The electronic control circuitry of the electronic control box reads the code and sets the device to perform a corresponding function selected from heating, cooling, electrical testing and visible light generation. In this embodiment, one button (FIG. 1) is used to start and stop the identified operation.

Still referring to FIG. 3, in some embodiments each probe includes a resistor having a pre-determined resistance that can be detected by the the electrical control circuitry, thereby identifying the type of each probe.

Additionally or alternatively, the electronic control circuitry may include an optical transmitter and an optical receiver, or a plurality of pairs of optical transmitters and receivers, separated by a gap defined by the probe holder. Also, each probe includes spaced-apart blockages and/or apertures in pre-determined patterns (e.g. binary code). When a probe is inserted into the gap of the probe holder, some free-space optical paths through the gap are blocked and some are not blocked in accordance with the pre-determined pattern that identifies the type of probe.

Additionally or alternatively, each probe may be marked by a bar code that is read by the electronic control circuitry upon insertion of the probe into the probe holder, such that the type of probe becomes identified.

In some embodiments in which the probe or type of probe is automatically identified, the electronic control circuitry is operable to receive user input directing the electronic control circuitry to override the automatic identification such that a given inserted probe is controlled by the electronic control circuitry as if it were of a different probe type. The different probe type may be user-selected, for example.

Exemplary physical characteristics in accordance with some embodiments are provided in the table 1 below.

TABLE 1
Exemplary Physical Characteristics

| | | | | Measurement | | | |
|---|---|---|---|---|---|---|---|
| Req# | Parameter | Symbol | Description | min | target | max | Unit |
| P1 | Handpiece weight | — | Can be easily operated | — | 200 | 350 | gr |
| | | | by an average person. | | | | |
| P2 | Probe visible length | L₁ | | 20 | 30 | 40 | mm |
| P3 | Probe tip geometry | — | Refer to figure(s) herein | — | — | — | — |
| P4 | Handpiece length | L₂ | | | 120 | 150 | mm |
| P5 | Handpiece diameter | D | | 10 | 20 | 25 | mm |
| P6 | Wire (connection) length | L₃ | | 1.5 | 1.5 | 2 | m |

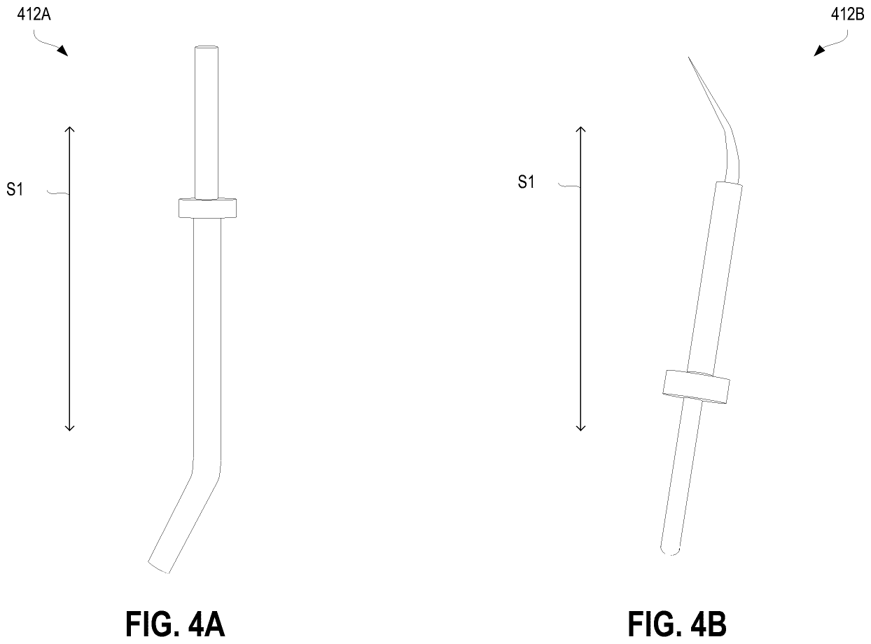

With reference to FIG. 4, in various embodiments the probe's shape may be straight, bent, pointed, or may have other geometries.

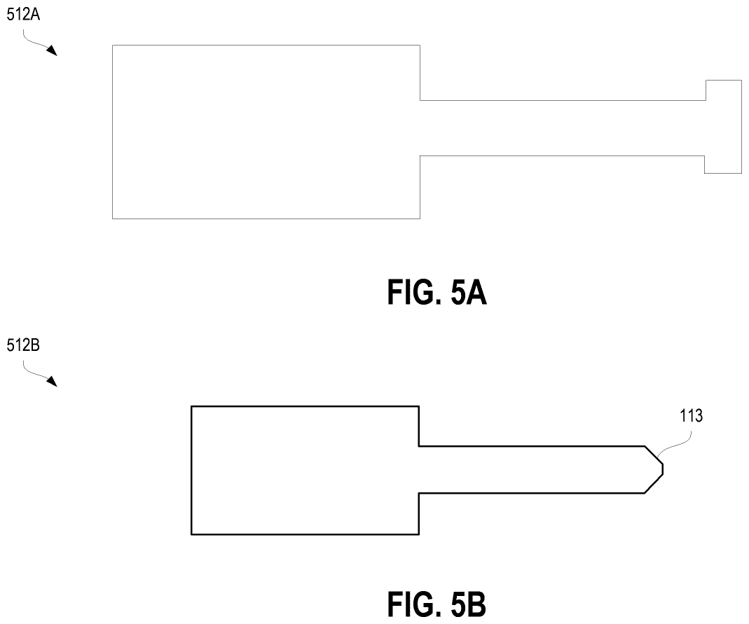

With reference to FIG. 5, in some embodiments the probe is a T-shaped probe having a T-shaped tip. Alternatively, in some embodiments the probe is an angled-tip probe having an angled tip.

Exemplary electrical characteristics in accordance with some embodiments are provided in the table 2 below.

TABLE 2
Exemplary Electrical Characteristics

| | | | | Measurement | | | |
|---|---|---|---|---|---|---|---|
| Req# | Parameter | Symbol | Description | min | target | max | Unit |
| E1 | Operating voltage | Vᵢₙ | DC voltage from external | 5 | TBD | 24 | v |
| | | | adaptor | | | | |
| E2 | Power consumption | W | | | TBD | | W |
| E3 | Physical buttons | C, H, E, L | momentary buttons on | 4 | 4 | 4 | — |
| | (Option 1) | | handpiece for hot, cold, | | | | |
| | | | Electrical and Light | | | | |
| E4 | Physical button | [symbol] | One momentary button on | 1 | 1 | 1 | — |
| | (Option 2) | | handpiece to start the | | | | |
| | | | operation | | | | |
| E5 | Power switch | s | Power On/Off on the | 1 | 1 | 1 | — |
| | | | Control box | | | | |
| E6 | Indicator | LED | RGB LED on the handpiece | — | — | — | — |
| | | | to show status of the | | | | |
| | | | device: | | | | |
| | | | Device not ready → BLINK | | | | |
| | | | Device ready → SOLID | | | | |
| | | | Hot → RED | | | | |
| | | | Cold → BLUE | | | | |
| | | | Electric → GREEN | | | | |
| | | | Light → WHITE | | | | |
| E7 | Control box weight | — | Can be easily carried and | — | — | — | — |
| | | | moved by an average | | | | |
| | | | person. | | | | |
| | Display | | 2 digit numeric display (or | | | | |
| | | | more) | | | | |

Exemplary safety and environmental characteristics in accordance with some embodiments are provided in the table 3 below.

TABLE 3
Exemplary Safety and Environmental Characteristics

| Req # | Parameter | Description |
|---|---|---|
| S1 | Anti choking | No component should break or get loose to go |
| | | inside the throat |
| S2 | No sharp edge | |
| S3 | No risk of | |
| | electric shock | |
| S4 | Bio | Probes and any other parts that goes inside the |
| | Compatible | mouth must be bio compatible |
| S5 | Hot and cold | avoid extreme hot or cold condition on the |
| | protection | tip of probe (time out, secondary switch, etc. . .) |
| S6 | Sterilizable | Can be placed in autoclave or wiped down |
| | | with standard cleaning solutions |

The handpiece in some embodiments includes a heating element operable to generate heat in response to applied electrical power. The temperature of the heating element is controlled by varying the voltage and electrical current that is applied to the heating element. Such generated heat is transferable to a thermally conductive probe installed at the handpiece. In variations of embodiments, the heating element is made of nichrome, other resistance-heating alloy material, other material, including other alloy material, having a high electrical resistance, or any combination thereof for example.

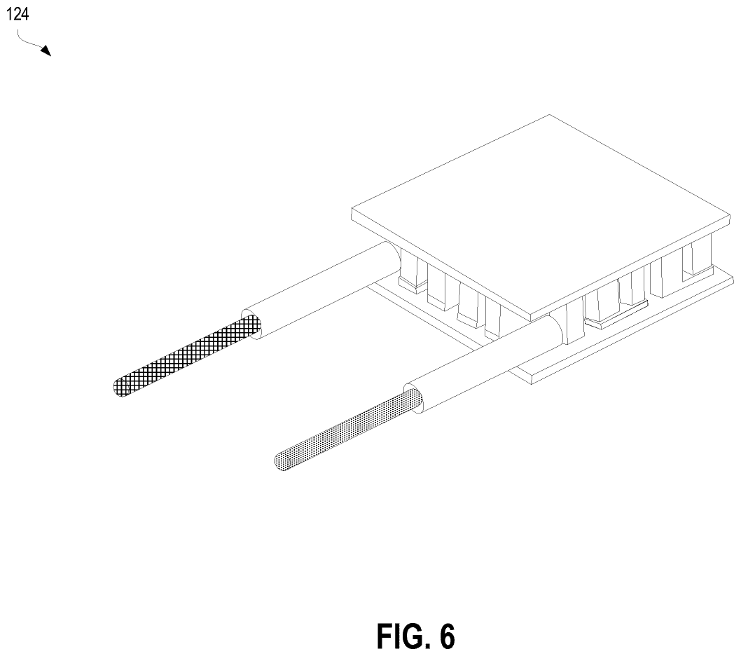

With reference to FIG. 6, the dental tool in some embodiments is operable to generate a hot or cold surface by using principle of a Peltier effect. In such embodiments, the dental tool includes a thermoelectrical device, which may be referred to as a Peltier that is an electronic semiconductor component that can act as an active heat pump. When the Peltier is connected to a voltage source, it transfers heat from one side to the other side of the Peltier.

In an exemplary embodiment, the Peltier thermoelectric device is made by “Custom Thermoelectric” company located in “11941 Industrial Park Road, Suite 5|Bishopville, Md. 21813”. The physical properties of the exemplary Peltier device are as follows:

Imax (amps)=6

Qmax (watts)=6.9

Vmax (volts)=2.1

dT Max (° C.)=67

Length (mm)=15

Width (mm)=15

Height (mm)=3.9

Part Number=01711-5L31-06CF

In some embodiments, the hot and cold system of the dental tool includes a Peltier, a constructed heatsink, a fan, a probe holder, thermally conductive probes and an electronic circuit. The dental tool in some embodiments includes a heat pipe.

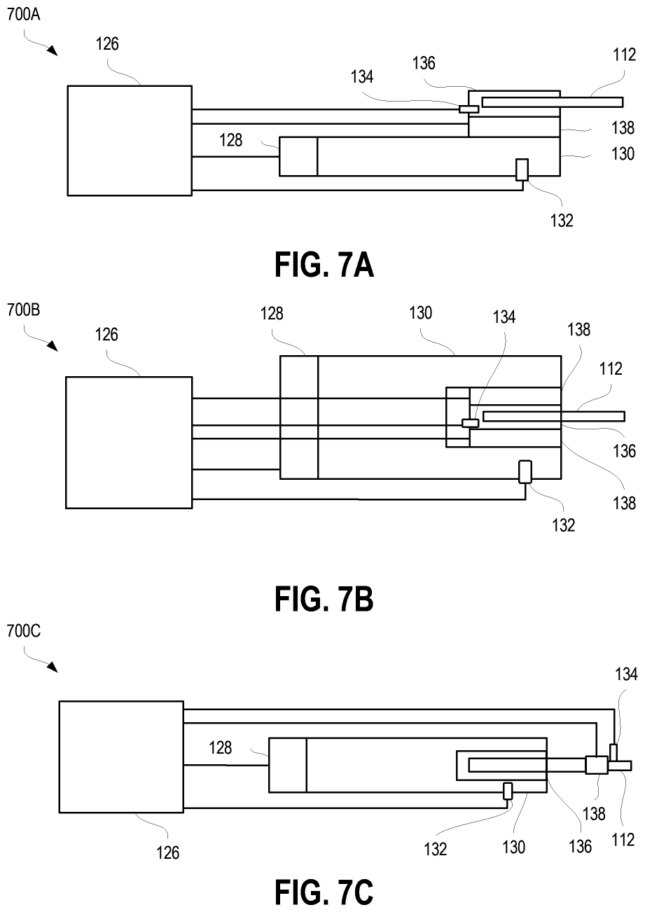

With reference to FIG. 7, the following is noted in respect of some and various embodiments:
An engineered heat sink is attached to one side of the Peltier to absorb and dissipate any unwanted heat.
A heat pipe is in thermal communication with one side of the Peltier to transfer heat energy away from the Peltier, such as by transferring heat energy toward the heat sink.
In embodiments having air cooling of the Peltier device, a fan at the heat sink circulates the air to cool the heat sink.
The fan may be a small fan.
The probe holder is made from copper or other conductive material which is securely attached to the opposite side of the Peltier.
The probe holder has a spring-loaded cavity to hold the probe tight and secure.
The probe is also made from copper or other conductive material, with different probes having different shapes and lengths for various functions.
Different probes, or different types of probes, may be color-coded to facilitate the correct selection of each probe that is used.
A dedicated electronic circuit includes a microprocessor, the Peltier and fan driver(s), sensors and indicators.
The electronic circuit receives data from a temperature sensor and applies a controlled voltage and a controlled current to the Peltier to control the temperature.
When required, the electronic circuit changes the voltage polarity to switch between hot and cold.
A RGB (Red, Green, Blue) LED (Light-Emitting Diode) light indicates the status of operation of the dental tool.
A display, such as a LCD (Liquid Crystal Display), displays information and data such as operational mode of the dental tool.
The microprocessor monitors the temperature of the probe and compensates for any heat loss due to emission or heat load.
The microprocessor controls the speed of the fan to optimize any tradeoff between heat dissipation and fan noise.

Exemplary thermal characteristics in accordance with some embodiments are provided in the table 4 below.

TABLE 4
Exemplary Thermal Characteristics

| | | | | Measurement | | | |
|---|---|---|---|---|---|---|---|
| Req# | Parameter | Symbol | Description | min | target | max | Unit |
| T1 | Probe COLD | T_{c} | Temperature | −5 | −7.5 | −10 | ° C. |
| | temperature | | measurement at tip of | | | | |
| | | | probe within test period | | | | |
| | | | on a tissue at 37° C. | | | | |
| T2 | Operation ready time @ | t_{1c} | When probe is at room | 10 | 12.5 | 15 | s |
| | COLD | | temperature | | | | |
| T3 | COLD test period | t_{2c} | Duration of cold test | 4 | 4 | 5 | min |
| T4 | Probe HOT temperature | Tₕ | Temperature Measured | 90 | 95 | 100 | ° C. |
| | | | within test time | | | | |
| T5 | Operation ready time @ | t₁ₕ | When probe is at room | 10 | 12.5 | 15 | s |
| | HOT | | temperature | | | | |
| T6 | HOT test period | t₂ₕ | Duration of hot test | 4 | 5 | 5 | min |
| T7 | Test interval | tᵢ | Time between 2 | 30 | 30 | — | min |
| | | | consecutive hot or cold | | | | |
| | | | test | | | | |
| T8 | Handpiece temperature | tₕₚ | Allowable temperature of | — | 37 | 40 | ° C. |
| | | | handpiece | | | | |

Thus, the present invention constitutes a novel and improved method to apply heat or cold stimuli to the tooth surface while reducing risk of tooth and patient injury. The method utilizes a Peltier thermoelectric device which can alternately generate either heat or cold. The present inventive method of applying thermal stimuli to the pulpal provides many advantages. For example, both cold and heat temperatures can be controlled, thereby reducing the risk of accidental patient injury. Furthermore, the Peltier thermoelectric device allows for switching between heating and cooling stimuli instantly with rapid changeover from a hot temperature to a cold temperature or vice versa, thereby reducing the testing time and accordingly reducing patient anxiety. This has the effect of reducing the likelihood of a patient's premature response to the stimuli due to anxiety and the anticipation of pain.

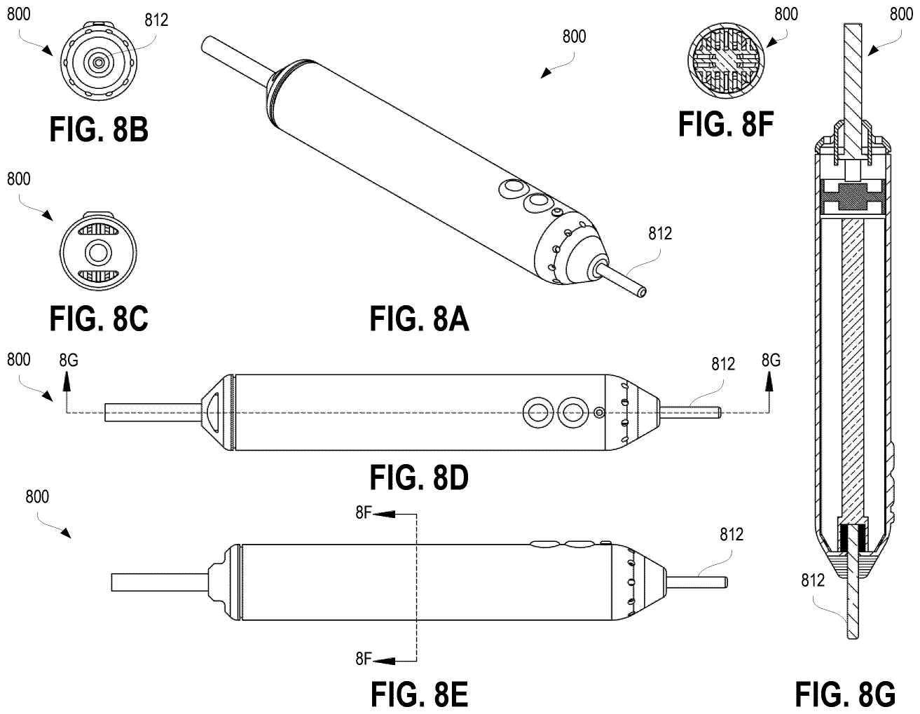

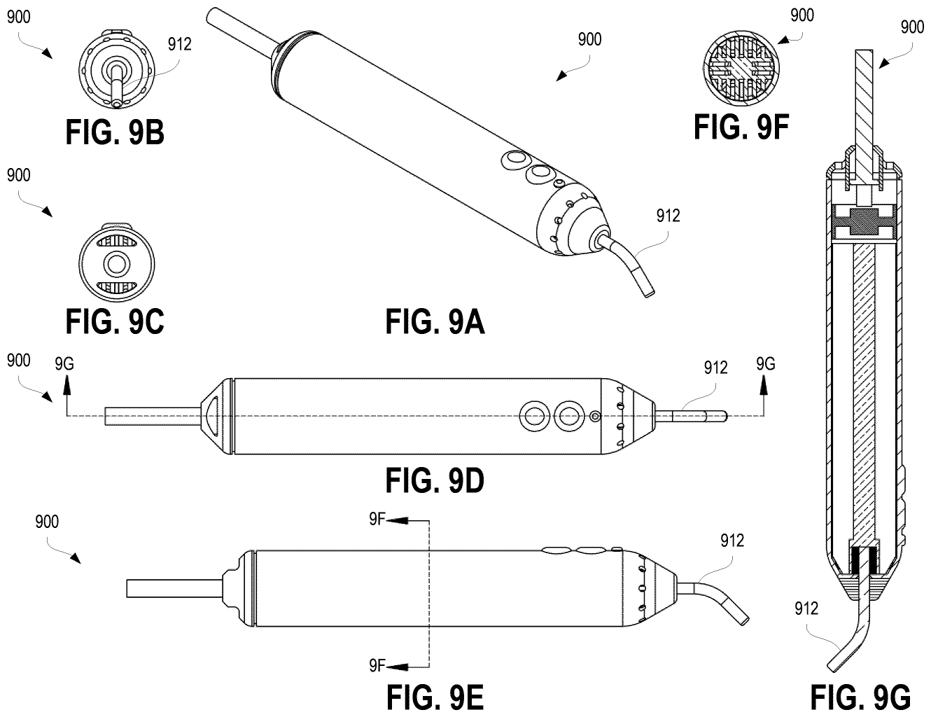

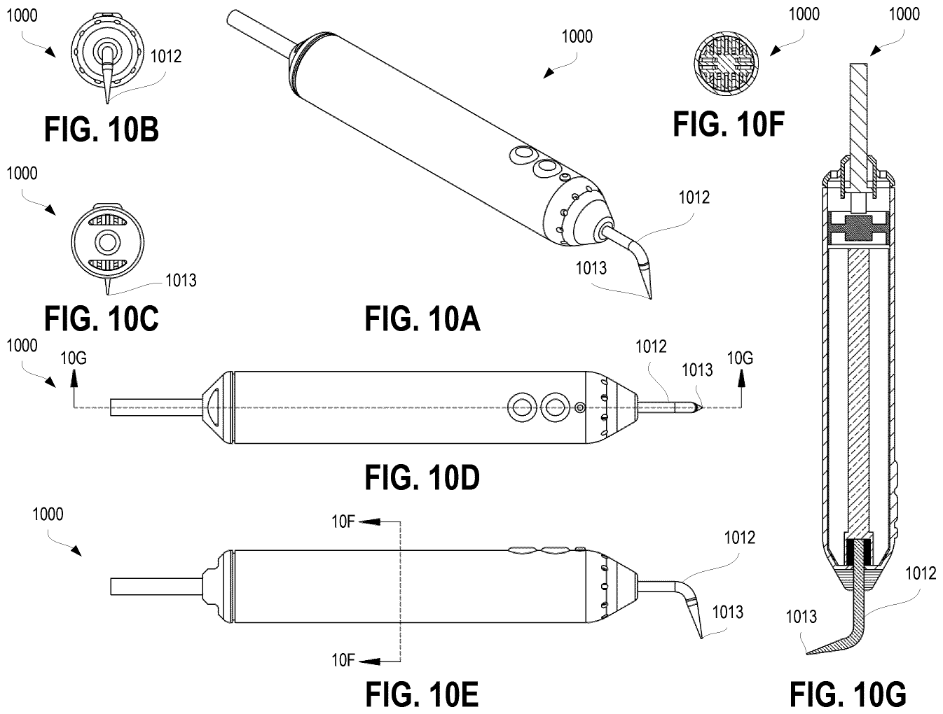

With reference to FIGS. 4 and 8 to 10, heat-conductive metal probes are used to transfer heat from or to the Peltier thermoelectric device to or from the tooth. The probes are attached to one side of the Peltier device by the probe holder which, in variations of embodiments, uses a mechanical lock feature, magnets, or a combination of both. In some embodiments, the probes come in different shapes to accommodate the various teeth of both jaws. In some embodiments, disposable probes are detachable from and attachable to the probe holder so as to be in thermal communication with the Peltier device via the probe holder. The disposable probes in some embodiments are single-use probes. In some embodiments, reusable probes are detachable from the Peltier device in some embodiments to allow the probes to be sterilized, such as by using an autoclave for example. Probes with pointed tips particularly allow for applying heat/cold to a tooth at a desired point on the tooth, thereby making thermal vitality tests on a tooth with full coverage crown more reliable for example. In some embodiments, the probe and/or probe tip is dimensioned for contacting a tooth surface under an installed crown or other dental restoration. In some embodiments, the probe and/or probe tip is dimensioned for placement between a tooth and a dental restoration of that tooth, such as a crown for example.

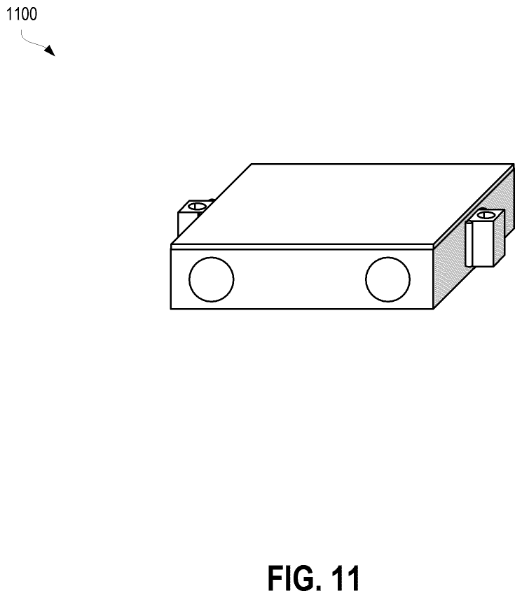

With reference to FIG. 11, the Peltier device generates excessive heat on its hot side. Different techniques can be used to dissipate the excessive heat. In some embodiments, the dental tool includes a water-cooling system having an electric fan, cooling radiator (not shown), water block (shown in FIG. 11), water pump (not shown), and tubing (not shown). The water-based cooling system is used to dissipate excess heat generated by hot surface of Peltier device. The water block is attached to the hot side of the Peltier device. Constant water flow in the water block prevents the hot side of the Peltier device from overheating. In some embodiments, a liquid coolant is circulated in addition or in substitution to circulating water. Water has a very high specific heat capacity allowing it to absorb heat energy generated by the Peltier device on its hot side. A heat sink can also be used at the hot side of the Peltier device to transfer heat to water. If the water temperature rises, it will not be as effective in dissipating the excessive heat from the hot side of the Peltier device. Thus, in order to keep the water temperature sufficiently low, a fan-and-radiator based water-cooling system in implemented in some embodiments.

Components of the cooling system are connected through the tubing, such as flexible silicone or other rubber tubing, to form a closed circuit. The water pump pushes water into the cooling radiator and then to the water block. Water, then is transferred back from the water pump to the water pump and cycle continues. The electric fan is attached to the cooling radiator, keeping the water exiting the radiator at a continuously low temperature.

An exemplary 8-Pipe Aluminum Heat Exchanger Radiator with Fan (not shown) that is useable in embodiments of the present invention is sold by “Clyxgs” at the Amazon™ website. The specifications of the exemplary radiator and fan are as follows:

Fitting Thread: G¼ inch;

Fin density: 18 lines per inch

Weight: 106 g

Material: Aluminum

Mouth: 9.5 (D) mm

Longer Screw Size: 31×3 mm (L*D);

Length (mm)=105

Width (mm)=80

Height (mm)=37

Fit ID Tube Inner Diameter: 8-12 mm

Fit ID Tube Outer Diameter: 9.5 mm

Shorter Screw Size: 7×3 mm (L*D)

An exemplary water block (FIG. 11) that is useable in embodiments of the present invention is made by “Custom Thermoelectric” company located in “11941 Industrial Park Road, Suite 5|Bishopville, Md. 21813”. The properties of the exemplary water block are as follows:

Part Number: WBA-0.60-0.28-CU-01

Length (mm)=0.59

Width (mm)=0.59

Height (mm)=0.28

Usable area=15×15 mm

CNC machined from solid Copper 110.

An exemplary water pump (not shown) that is useable in embodiments of the present invention is made by “Shenzhen Tomtop Technology Co., Ltd.” Located in 4/F, No. A3 building HeKan Industrial Pa, Shenzhen, Guangdong, China. The specifications of the exemplary water pump are presented in the following:

Pump brand: Decdeal

Voltage: DC 12V

Max. Power: 5 W

Max. Current: 416 mA

Noise: Less than 35 dB

Outer Dia. of Inlet/Outlet: 8.5 mm

Inner Dia. of Inlet/Outlet: 6 mm

Max. Liquid Temperature: 60° C.

Cable Length: 100 cm

Product Size: 59.5*49*42 mm

Product Weight: 67 g

In some embodiments, a direct current (DC) power supply is used to power the cooling system and the Peltier device. An exemplary DC power supply with adjustable current and voltage is made by “Tech Instrumentation” company located in 750 E Kiowa Ave. Elizabeth, Colo. 80107-2029, USA. The properties of the exemplary power supply are as follows:

Output voltage: DC 0-30V Continuous adjustable

Output DC Current: 0-10 A

Overall efficiency: >89%

Voltage regulation: CV 0.01%+3 mV, CC<0.2%+6 mA

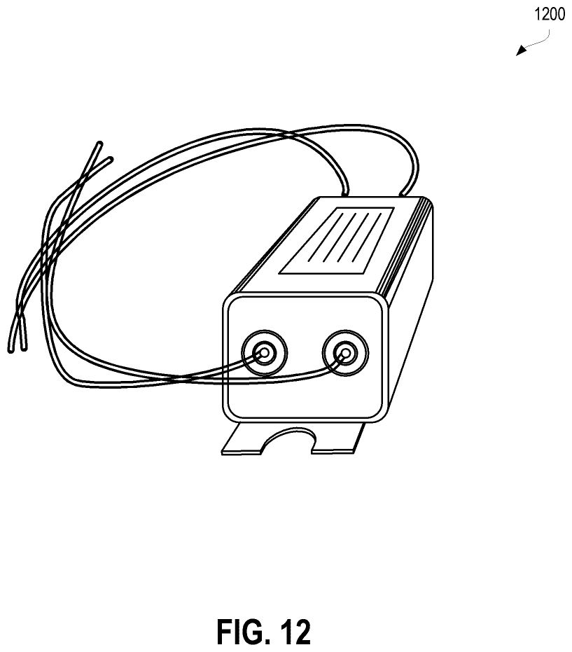

With reference to FIG. 12, a DC power converter is used to adjust the voltage and current fed into the Peltier device relative to the voltage and current fed into the cooling system. The electric fan and water pump of the water cooling system require higher voltage and current to function in comparison to the voltage and current requirements of the Peltier device. The cold/hot temperature generated by the Peltier device is controlled by changing the voltage and/or current supplied to the Peltier device. The heat/cold that is transferred to the metallic probes can then be adjusted by changing the temperature produced by the Peltier device. Also, changing the contact area of a given probe (e.g. by selecting a different removably attachable probe) changes the effect on the tooth. In different embodiments, different surface areas of the Peltier device will produce different temperature effects.

In embodiments having a water cooling system and also in embodiments having an air fan and heatsink cooling system, when electrical current flows through the Peltier device and separately through the water or air cooling system, the temperature on the cold side of the Peltier device drops to a controllable cold, possibly including a freezing, temperature (e.g. about −15 C), typically within a few seconds, while the temperature on the hot side of the Peltier device remains low due to the cooling system.

In at least some of the water-cooled embodiments, the water block does not completely cover the hot side of the Peltier device. For example, approximately 1.5 mm of the ceramic plate of the Peltier device can remain uncovered. The uncovered portion of ceramic plate can be used for thermal communication between the hot side of the Peltier device and a heating probe. In such embodiments, heating and cooling probes are attachable thermally to the hot and cold sides of the Peltier device to transfer heat and cold to a patient's tooth, respectively. The probes can be placed in thermal communication with the ceramic plates of the Peltier device via magnets and/or mechanical lock, for example.

To transfer heat/cold from the Peltier ceramic plates to a patient's tooth, heat conductive (e.g. copper) probes can be used. Different heating probes can be made in different forms and shapes for contact with the patient's tooth at different tooth locations within the oral cavity, for example.

An exemplary 12V to 6V, 6 A, and 36 W Power Converter that is useable in embodiments of the present invention is made by “Shenzhen Shenhuo Trade Co Ltd” company located in Room 408 Block C, No. 339 Bulong Road, Longang District, Shenzhen, Guangdong, China. The properties of the exemplary power converter are as follows:
Part number: SMAKN-1224-6-36 W
Input voltage: DC:12V (Wide Voltage 8.5V-28V).
Output voltage: DC: 6V
Output current: 6 A
Power: 36 W
Length (mm)=43
Width (mm)=25
Height (mm)=22
Protection: Input instant high voltage, output over-current, output short circuit, chip overheating protection.

Exemplary flexible silicone rubber tubing that is useable in embodiments of the present invention is made by “uxcell” located in Unit 1111, 11/F, Tower 2 Metroplaza, No. 223 Hing Fong Road, Kwai Fong, China. The properties of the exemplary tubing are as follows:

Silicone rubber tube—will withstand repeated use

Size (mm): 4×6 (Inner Dia*Outer Dia)

Wall Thickness (mm)=1 mm

Operating Temperature: −60 to +220 C Degree

Hardness: 65

In some embodiments, the silicone rubber tube is selected to be transparent or translucent so as to permit visual inspection of fluid or fluid flow within the tube.

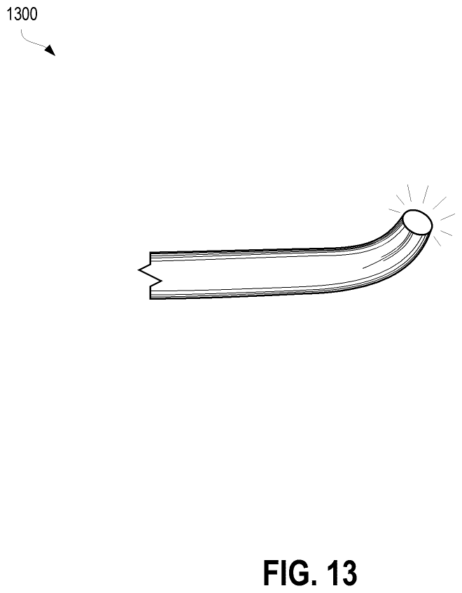

With reference to FIG. 13, the probe in some embodiments emits visible light. In exemplary embodiments, the light is powered by a high intensity Light Emitting Diode (LED) and transmitted through a focused glass fiber-optic element.

Exemplary optical characteristics in accordance with some embodiments are provided in the table 5 below.

TABLE 5
Exemplary Optical Characteristics

| | | | Des- | Measurement | | | |
|---|---|---|---|---|---|---|---|
| Req# | Parameter | Symbol | cription | min | target | max | Unit |
| | Light wavelength | | | 400 | 550 | 700 | nm |
| | Intensity | | | 3000 | 5000 | 5000 | Lux |
| | Diameter | | | 2.5 | 3 | 3.5 | mm |

In some embodiments, the fiber-optic transillumination probe, the electrically conductive probes, and the thermally conductive probes are interchangeable with each other and can be separately mounted on the mentioned handpiece. In some embodiments, the transillumination probe and the electrically conductive probes are attachable to the handpiece via a mechanical lock (e.g. of a probe holder). In some embodiments, the electrically conductive probes and the thermally conductive probes are made out the same electrically and thermally conductive material (e.g. copper). The transillumination probe can be used, such as by holding the transillumination probe against the crown of a tooth, to detect cracks and/or fractures on the crown of the tooth.

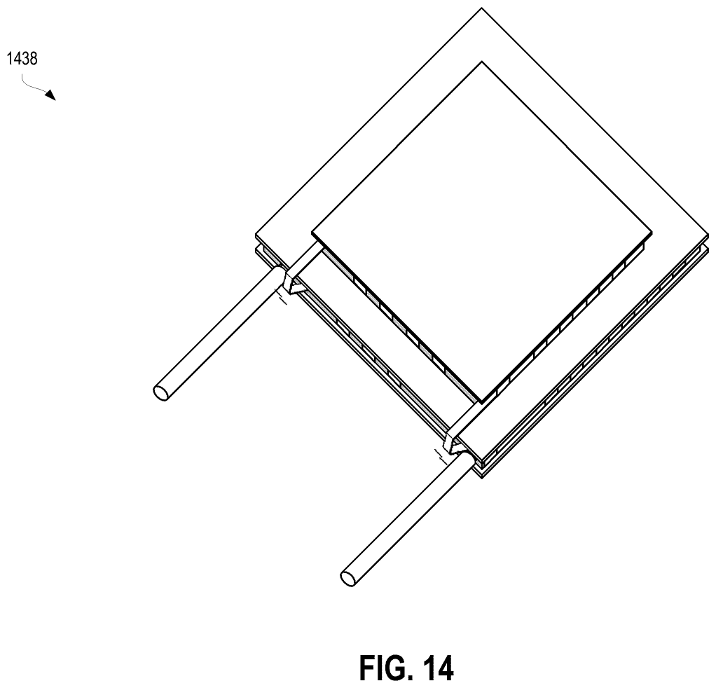

With reference to 14, the dental tool in some embodiments includes a multiple-stage thermoelectrical device, such as the two-stage thermoelectric device shown in FIG. 14. The use of a multi-stage thermoelectric device advantageously facilitates reaching colder temperatures, for example as shown by the exemplary thermal characteristics shown in the table 6 below.

TABLE 6
Exemplary Thermal Characteristics (Multi-Stage Thermoelectric Device)

| | | | | Measurement | | | |
|---|---|---|---|---|---|---|---|
| Req# | Parameter | Symbol | Description | min | target | max | Unit |
| T1 | Probe COLD | T_{c} | Temperature | −10 | −15 | −20 | ° C. |
| | temperature | | measurement at tip of | | | | |
| | | | probe within test period | | | | |
| | | | on a tissue at 37° C. | | | | |
| T2 | Operation ready time @ | t_{1c} | When probe is at room | 10 | 60 | 90 | s |
| | COLD | | temperature | | | | |
| T3 | COLD test period | t_{2c} | Duration of cold test | 4 | 4 | 5 | min |
| T4 | Probe HOT temperature | Tₕ | Temperature Measured | 60 | 65 | 70 | ° C. |
| | | | within test time | | | | |
| T5 | Operation ready time @ | t₁ₕ | When probe is at room | 10 | 12.5 | 15 | s |
| | HOT | | temperature | | | | |
| T6 | HOT test period | t₂ₕ | Duration of hot test | 4 | 5 | 5 | min |
| T7 | Test interval | tᵢ | Time between 2 | 30 | 30 | — | min |
| | | | consecutive hot or cold | | | | |
| | | | test | | | | |
| T8 | Handpiece temperature | tₕₚ | Allowable temperature of | — | 37 | 40 | ° C. |
| | | | handpiece | | | | |

As shown in the table 6 above, the use of a multi-stage thermoelectric device (FIG. 14) permits the dental tool to provide a temperature at the probe tip as cold as −20 degrees celsius.

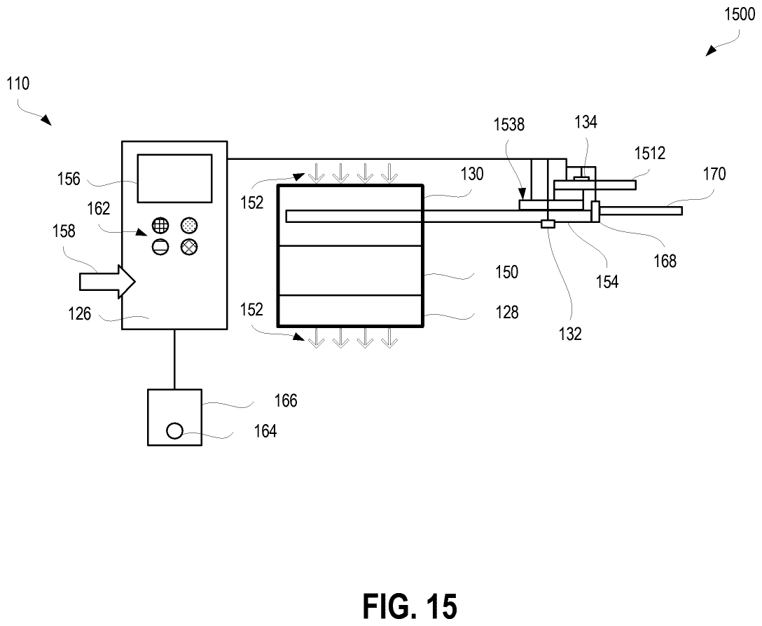

With reference to FIG. 15, the dental tool according to a fourth embodiment includes a multi-stage thermoelectric device (FIG. 14) and a heat pipe for high-efficiency of heat absorption from the multi-stage thermoelectric device and for transferring such heat energy to a heatsink for heat dissipation. In such embodiment, the heat pipe is disposed in thermal communication between one side of the multi-stage thermoelectric device and the heatsink.

The dental tool shown in FIG. 15 includes a fixed probe that is not readily detachable nor reusable. The fixed probe advantageously provides superior heat transfer from the multi-stage thermoelectric device in comparison to a removably attachable probe. In variations, the probe may be straight or include one or more angled sections. Additionally or alternatively, the probe tip may have various shapes such as being rounded or T-shaped for example. In some embodiments, the dental tool is operable to receive a probe-tip adaptor for altering the shape or direction of the probe tip. The dental tool may include one or more user-selectable probe-tip adaptors having different dimensions.

The dental tool of FIG. 15 includes a removable light pipe that can be readily removed by the user when not in use. In some embodiments, the light pipe is adjustable such that when the light pipe is installed its tip can be pointed in a desired direction within a range of light directions. The dental tool according to the fourth embodiment typically produces white light as shown in FIG. 15.

The dental tool of FIG. 15 includes electronic circuitry for controlling operations of the dental tool. Such electronic circuitry may include any combination of hardware electronics, computing devices such as a CPU (Central Processing Unit) and/or computing memory, computing software, firmware, or the like. The electronic circuitry is typically powered by a DC (Direct Current) power source, and typically includes a handheld button for user actuation to generate user input to the electronic circuitry. In some embodiments, the dental tool includes indicators, such as LEDs (Light-Emitting Diodes) of any suitable colour for example, and/or a display such as a LCD (Liquid Crystal Display) or similar.

The dental tool of FIG. 15 includes a heatsink temperature sensor for sensing the temperature of the heatsink and/or the heat pipe. The dental tool of FIG. 15 includes a probe temperature sensor for sensing the temperature of the fixed probe or variation thereof. Additionally or alternatively, other sensors (not shown) may be included in the dental tool.

While FIG. 15 shows the combined use of the multi-stage thermoelectric device, the heat pipe, the fixed probe, the removable light pipe, the heatsink sensor, and the probe sensor, in variations of embodiments each of these features of FIG. 15 may be separately employed, such as in separate embodiments for example, or employed in any combination thereof.

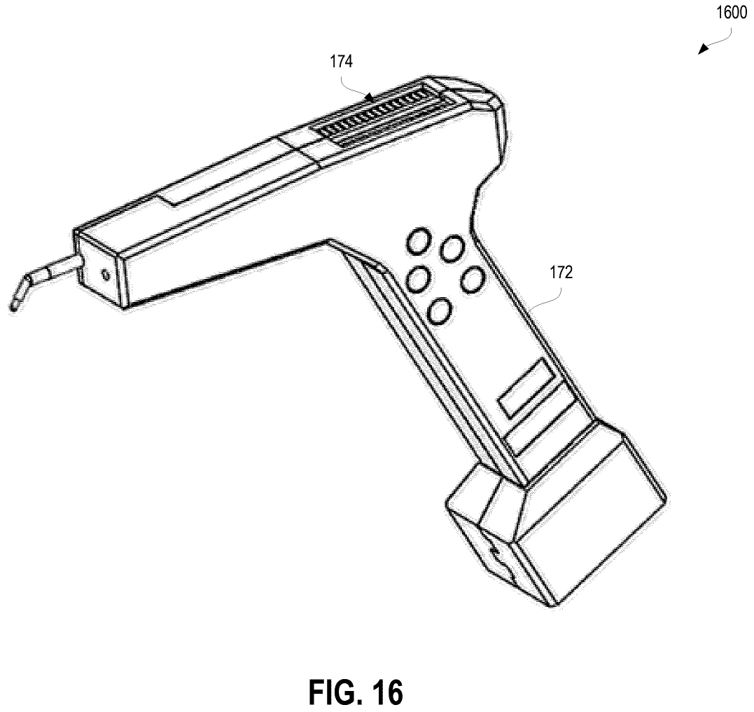

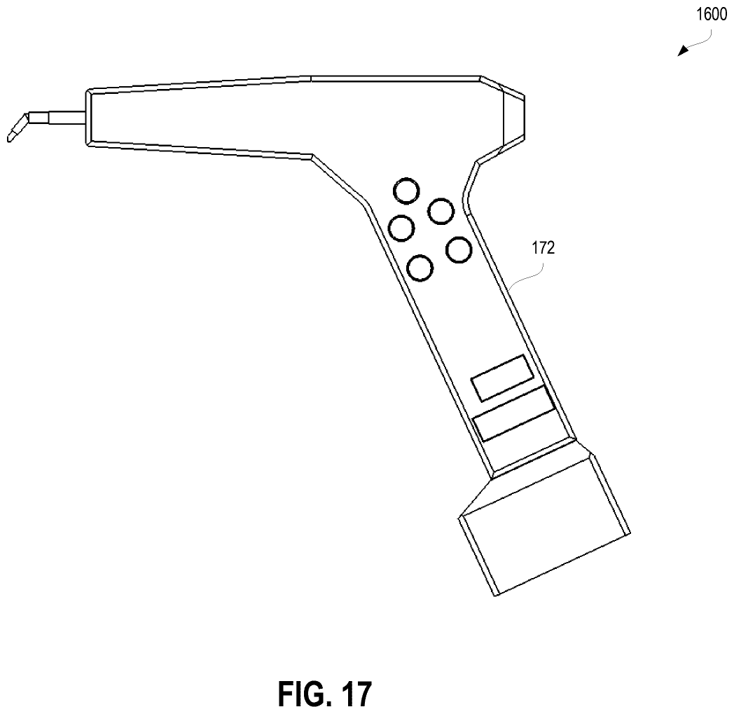

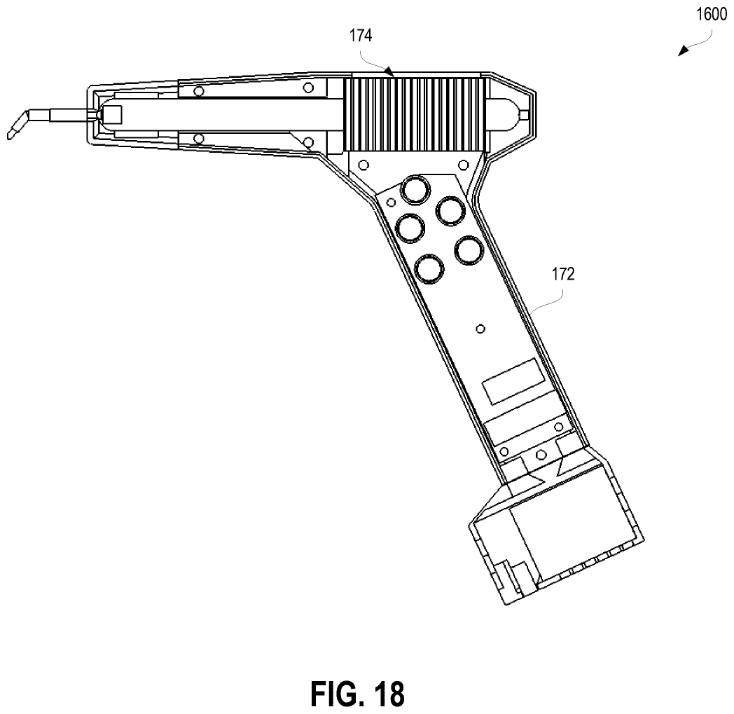

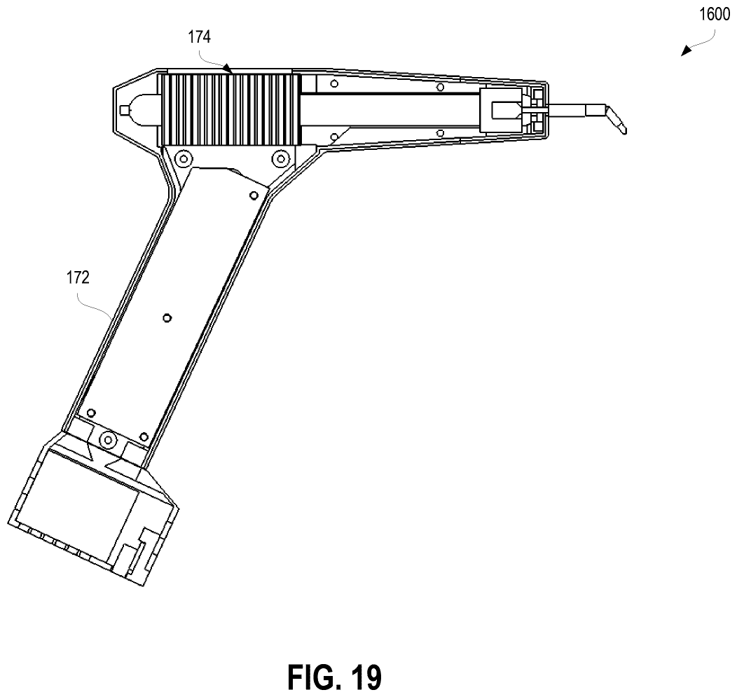

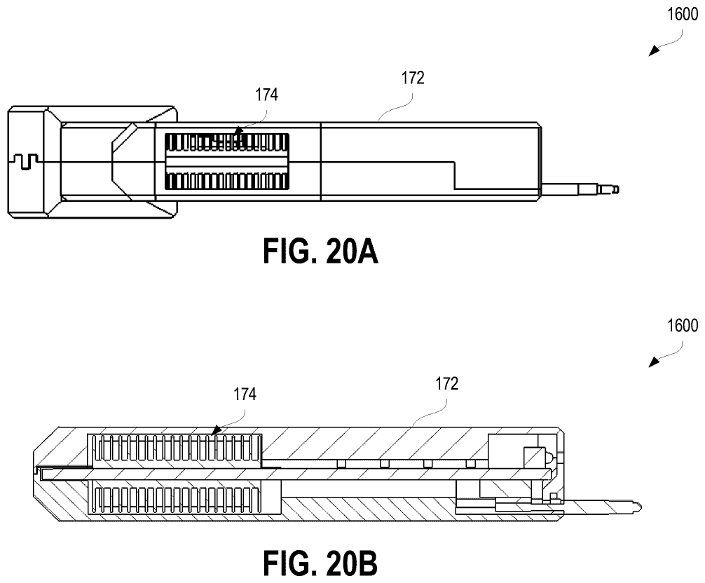

Referring to FIGS. 16 to 21, the dental tool according to the fourth embodiment or variations thereof includes a gun-shaped housing for ease of use. The gun-shaped dental tool includes air vents for airflow associated with the heat sinking features (FIG. 15) of the gun-shaped dental tool.

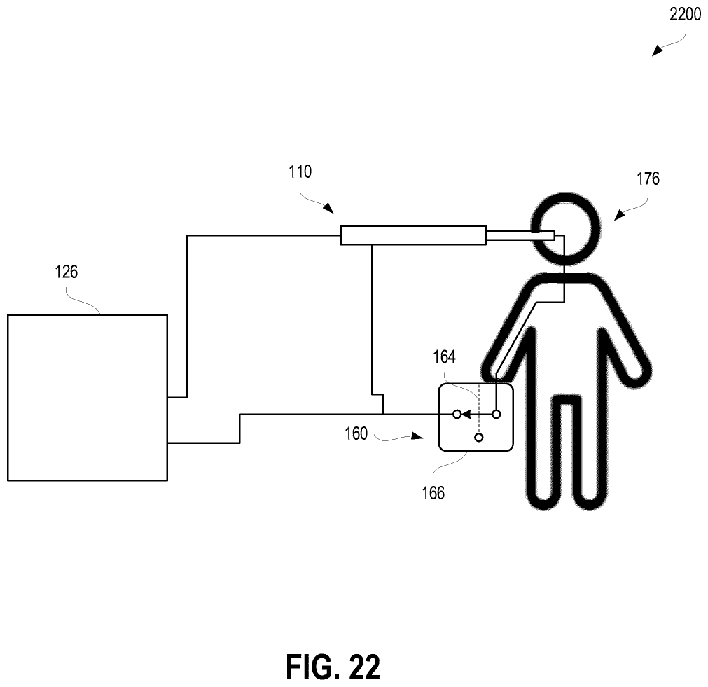

With reference to FIGS. 15 and 22, the gun-shaped dental tool in some embodiments includes the display and/or indicators. Preferably, the display and indicators are located toward the rear of the dental tool in proximity to each other for convenient viewing by a user.

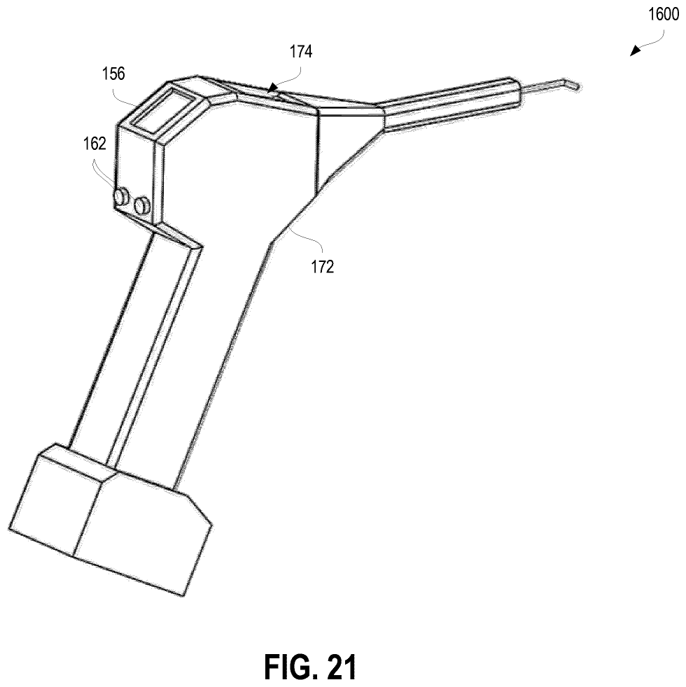

In variations of embodiments, all or part of the electronic control circuitry can be housed within the gun-shaped housing or implemented as part of a separate control console in electical communication with the gun-shaped portion of the dental tool. In variations, the display can be integrated into the gun-shaped housing as shown in FIG. 21, and/or included in the separate control console. In embodiments having a console, the console may be operable to store accessories such as replaceable probes and/or probe-tip adapters, for example.

In embodiments of the invention, the probe is mechanically secured to the heat pipe, heat sink, or other heat conduit. In embodiments having a fixed probe, the fixed probe at its internal end is wrapped in an aramid-based material, such as Kevlar™, and an epoxy or other adhesive is employed to maintain the material in place at the internal end of the fixed probe.

In embodiments of the invention, thermal insulation is present between the hot and cold regions of the dental tool associated with the probe and heatsinking. For example, the exterior portion of the probe is thermally and electrically insulated to better maintain the desired temperature at the probe tip and to electrically protect tissue of a dental patient in contact with the probe.

With reference to FIG. 22, the dental tool is suitable for use in an electrical pulp vitality test. Exemplary electrical pulp test characteristics in accordance with some embodiments are provided in the table 7 below.

TABLE 7
Exemplary Electrical Pulp Test Characteristics

| | | | | Measurement | | | |
|---|---|---|---|---|---|---|---|
| Req# | Parameter | Symbol | Description | min | target | max | Unit |
| EP1 | Probe Voltage | Vₚ | | | | | V |
| EP2 | Probe Current | Cₚ | | | | | mA |
| EP3 | Probe Frequency | fₚ | | | | | Hz |
| EP4 | Test duration | tₚ | | 10 | 15 | 20 | s |
| EP5 | Variable parameter | X | Any parameter that | | | | |
| | | | should change to perform | | | | |
| | | | the test (Voltage, Current, | | | | |
| | | | Frequency) | | | | |

Referring to FIG. 22 and table 7 above, the voltage (V), current (C), and frequency (f) can be measured and determined. In some embodiments, the following settings are employed: Low, medium, and high. In some embodiments, the electrical current increases gradually whenever possible. In use, the electrical test probe can be placed against a dried surface of the crown and an electric pulse sent through the tooth's pulp to determine pulp vitality status. For example, the patient may press the pushbutton of the patient-held handpiece having an electrically conductive enclosure in contact with the patient's hand, thereby signaling to the electronic control circuit to stop the application of electrical power via the electrical test probe to the patient's tooth. In some embodiments, an audio signal (e.g. buzzer) is activated when the patient presses the handheld pushbutton of the patient's handpiece. In some embodiments, a timer is stopped when the patient presses their handheld pushbutton.

Advantageously, the dental tool of the present invention, which in some embodiments may be referred to as a Diagnostic Pulp Tester (DPT), facilitates pulpal diagnosis by combining heat, cold, transillumination, and electric pulp tests into one multi-function tool that is operable to employ different removably attachable probes. The dental tool can be used to generate heat and cold in a short period of time in a dental setting.

The generated head/cold can be used in different dental applications such as heating sodium hypochlorite, and cryotherapy. For example, a heating probe of some embodiments is dimensioned for placement inside a root canal during sodium hypochlorite irrigation to activate the sodium hypochlorite and increase its ability to dissolve pulpal tissue. Such heating probe is preferably needle-like thin (e.g. as thin as 0.1 mm diameter in some cases). In some embodiments, a cooling probe can be momentarily placed against the soft tissue at a desired injection site prior to injecting so as to reduce needle-penetration pain. Such cooling probe applied to the injection site preferably has a blunt, rounded tip for patient comfort. The temperature of such cooling probe is preferably at or near zero degrees Celsius such that applying the cooling probe to the injection site provides temporary topical anesthesia without causing tissue necrosis.

Advantageously, the dental tool of the present invention in some embodiments involves the use of a Peltier thermoelectric device to generate heat and cold for performing thermal pulp vitality testing. This method advantageously provides a superior method to thermal pulp test a tooth and to diagnose pulpal pathology status. Such thermal pulp test device in some embodiments includes a Peltier thermoelectric device, a cooling system, interchangeable metal probes, and a power supply. In some embodiments, the thermal pulp test makes use of the second handpiece held by the patient, such that an audio signal is activated, a timer is stopped, other functions performed, or any combination thereof for example occurs when the patient presses the pushbutton of the patient's handpiece.

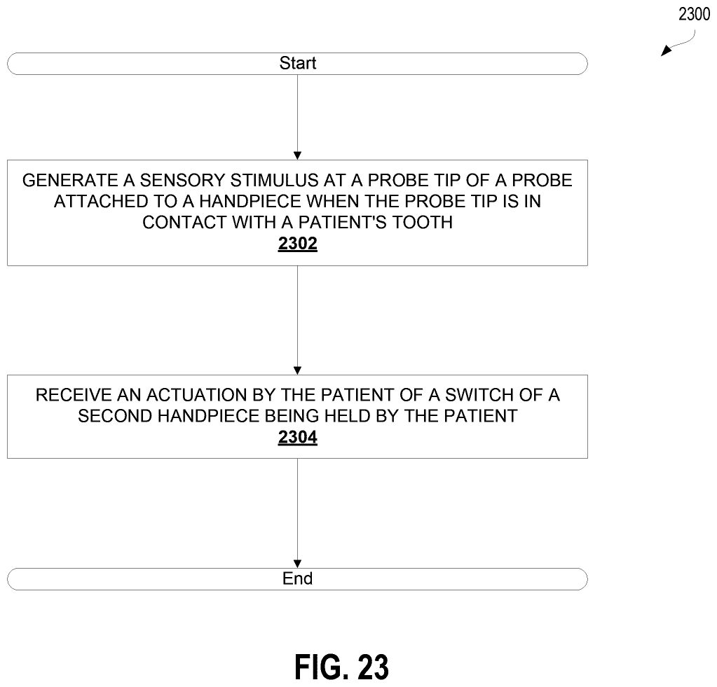

Referring to FIG. 23, an exemplary method of operation of the dental tool in accordance with various embodiments is shown. When power is being supplied to the electronic control circuitry, and the selected switch of the handpiece is actuated, then the electronic control circuitry generates a sensor stimulus at the probe tip of the probe that is attached to that handpiece. The probe tip is placed in contact with a patient's tooth or other dental feature within the oral cavity. In accordance with the dental procedure and typically under instruction of a dental professional, the patient experiences the stimulus and decides to actuate a switch of a second handpiece being held by the patient. At a subsequent step in the method, the electronic control circuitry receives the actuation by the patient of the switch of the second handpiece being held by the patient. Other method steps not shown in FIG. 23 may occur before, during or after each of the method steps shown in FIG. 23.

While embodiments of the invention have been described and illustrated, such embodiments should be considered illustrative of the invention only. The invention may include variants not described or illustrated herein in detail. Thus, the embodiments described and illustrated herein should not be considered to limit the invention as construed in accordance with the accompanying claims.

What is claimed is:

1. A dental tool having an electronic circuit, the dental tool comprising:
(a) a first handpiece including a first switch;
(b) a probe attachable to the first handpiece, the electronic circuit being operable to generate a sensory stimulus at the probe in response to actuation of the first switch; and
(c) a second handpiece including a second switch, the second handpiece being operable to transmit to the first handpiece a signal for controlling the sensory stimulus in response to actuation of the second switch.

2. The dental tool of claim 1 wherein the first handpiece is operable to generate the sensory stimulus selected from the group consisting of a thermal stimulus, an electrical stimulus, and a visual stimulus.

3. The dental tool of claim 1 wherein the probe defines a probe tip, the first handpiece comprises a thermoelectric device for generating the sensory stimulus as a thermal stimulus, the dental tool being operable to maintain the probe tip at a specifiable temperature in response to the actuation of the first switch.

4. The dental tool of claim 3 wherein the electronic circuit is operable to control electrical power supplied to the thermoelectric device, the second handpiece being operable to transmit the signal such that the electronic circuit stops supplying the electrical power to the thermoelectric device.

5. The dental tool of claim 3 wherein the first handpiece further comprises a heatsink in thermal communication with the thermoelectric device, and a fan operably coupled to the electronic circuit to generate airflow at the heatsink.

6. The dental tool of claim 5 wherein the first handpiece further comprises a heat pipe for conveying thermal energy between the thermoelectric device and the heatsink.

7. The dental tool of claim 5 wherein the first handpiece further comprises an air duct for conveying the airflow between the fan and the heatsink.

8. The dental tool of claim 3 wherein the thermoelectric device is a multi-stage thermoelectric device.

9. The dental tool of claim 3 wherein the dental tool comprises a probe sensor operable to sense a temperature of the probe at the probe tip.

10. The dental tool of claim 2 comprising a plurality of said probes, each said probe being removably attachable to the first handpiece, the first handpiece comprising a detector for identifying a type of said each probe, the sensory stimulus being selected by the electronic circuit in response to the type identified by the detector.

11. The dental tool of claim 10 wherein the detector is operable to sense a pre-determined electrical parameter exhibited by said each probe when said each probe is attached to the first handpiece.

12. The dental tool of claim 10 wherein the detector is operable to sense a pre-determined optical parameter exhibited by said each probe when said each probe is attached to the first handpiece.

13. The dental tool of claim 12 wherein the detector comprises an optical source and an optical receiver.

14. The dental tool of claim 10 wherein at least one said removably attachable probe is operable to produce visible light for dental transillumination.

15. The dental tool of claim 1 wherein the second handpiece is operable to transmit the signal to cause a timer to be stopped in response to the actuation of the second switch.

16. The dental tool of claim 1 wherein the electronic circuit is operable to produce an audio signal in response to the actuation of the second switch.

17. The dental tool of claim 1 wherein the first handpiece has a gun-shaped housing.

18. A method of conducting a dental test on a tooth of a patient, the method comprising:
(a) generating by an electronic circuit a sensory stimulus at a probe tip of a probe attached to a first handpiece when the probe tip is in contact with the tooth; and
(b) receiving by the electronic circuit an actuation by the patient of a switch of a second handpiece held by the patient.

19. The method of claim 18 wherein step (a) comprises generating an electrical stimulus, the method further comprising:
(c) ceasing to generate the electrical stimulus in response to receiving the actuation.

20. A dental tool for conducting a dental test on a tooth of a human patient, the dental tool comprising:
(a) handpiece means for generating a sensory stimulus at the tooth; and
(b) second handpiece means for receiving an actuation by the patient of a switch.
